(19) United States
(12) Patent Application Publication
(10) Pub. No.: US 2025/0235658 A1
(43) Pub. Date: Jul. 24, 2025

(54) PERSONAL FREQUENCY APPARATUS

(71) Applicants: Pasquale Falotico, Markham (CA); Linda De Vellis, Markham (CA); Alicia Zadravec, Markham (CA)

(72) Inventors: Pasquale Falotico, Markham (CA); Linda De Vellis, Markham (CA); Alicia Zadravec, Markham (CA)

(21) Appl. No.: 18/415,726

(22) Filed: Jan. 18, 2024

(51) Int. Cl. A61M 21/00; G16H 20/90

(52) U.S. Cl. CPC A61M 21/00; G16H 20/90; A61M 2205/3592; A61M 2205/502; A61M 2210/083

(57) ABSTRACT

A personal frequency apparatus that is configured to provide a plurality of chakra frequencies for transmission thereof. The present invention includes a housing wherein the housing includes electronics operable to receive, store, transmit and manipulate data. The housing in a preferred embodiment is configured to be worn on the wrist of a user. The housing includes a graphical display unit wherein the graphical display unit provides an interface with the operational software of the present invention. The software of the present invention provides a selection of chakra frequencies. The chakra frequencies are transmitted through a frequency transmission module that is disposed within the housing. The frequency transmission module includes mechanical and/or digital elements that provide generation and transmission of the chakra frequencies. A user will select from a provided menu of chakra frequencies wherein ensuing selection the frequency will be transmitted for at least forty-five seconds.

### FIELD OF THE INVENTION

The present invention relates generally to meditation devices, more specifically but not by way of limitation, a personal device that is worn by a user wherein the device includes a graphical interface to select a chakra frequency and wherein the present includes a frequency emitter configured to emit the selected frequencies.

### BACKGROUND

The word chakra appears to first emerge within the Vedas, though not in the sense of psychic energy centers, rather as chakravartin, meaning the king who turns the wheel of his empire in all directions from a center representing his influence and power. The iconography popular in representing the chakras traces back to the five symbols of yajna, the Vedic fire altar: square, circle, triangle, half-moon and dumpling. The chakras are part of esoteric ideas and concepts about physiology and psychic centers that emerged across Indian traditions. The belief held that human life simultaneously exists in two parallel dimensions, one dimension being the physical body and the other dimension being psychological, emotional and mind. The second dimension is thought to be of energy, while the first dimension of the physical body is mass. The psyche or mind plane corresponds to and interacts with the body plane, and the belief holds that the body and the mind mutually affect each other. The second dimension consists of connected by nodes of psychic energy called chakra. The number of major chakras varied between various traditions, but typically ranged between four and seven. Buddhist teachings mention eight chakras and there is a complete yogic system for each of them.

The more common and most studied chakra system incorporates six major chakras along with a seventh center generally not regarded as a chakra. These points are arranged vertically along an axial channel. According to authorities, this system of six chakras plus the sahasrara (center) at the crown first appears in 11th-century Kaula work. The Chakras are traditionally considered meditation aids. The yogi progresses from lower chakras to the highest chakra blossoming in the crown of the head, internalizing the journey of spiritual ascent. The chakras are pierced by a dormant energy residing near or in the lowest chakra. Each chakra is believed to have a energy frequency and focus on production of these frequencies can assist with meditation practice and the engagement of chakra.

Accordingly, there is a need for a personal frequency apparatus that is configured to be worn by a user wherein the apparatus provides a selection of chakra frequencies for a user to choose and have transmission thereof from the apparatus.

### SUMMARY OF THE INVENTION

It is the object of the present invention to provide a personal frequency apparatus configured to provide selection and transmission of a plurality of chakra frequencies wherein the present invention in a preferred embodiment is worn on a user's wrist.

Another object of the present invention is to provide a frequency transmission apparatus configured to provide a plurality of frequencies to transmit for a user wherein the present invention includes a graphical interface for operation thereof.

A further object of the present invention is to provide a personal frequency apparatus configured to provide selection and transmission of a plurality of chakra frequencies wherein the housing of the present invention includes a frequency transmission module.

Yet a further object of the present invention is to provide a frequency transmission apparatus configured to provide a plurality of frequencies to transmit for a user wherein the present invention includes a software driven interface operable to provide a selection from a plurality of chakra frequencies.

Still another object of the present invention is to provide a personal frequency apparatus configured to provide selection and transmission of a plurality of chakra frequencies wherein an alternate embodiment of the present invention includes integration of the elements of the present invention into clothing.

An additional object of the present invention is to provide a frequency transmission apparatus configured to provide a plurality of frequencies to transmit for a user wherein the present invention can be programmed to emit a chakra frequency on an hourly basis.

Yet a further object of the present invention is to provide a personal frequency apparatus configured to provide selection and transmission of a plurality of chakra frequencies wherein the user of the present invention can select a frequency on demand for transmission by the apparatus of the present invention.

To the accomplishment of the above and related objects the present invention may be embodied in the form illustrated in the accompanying drawings. Attention is called to the fact that the drawings are illustrative only. Variations are contemplated as being a part of the present invention, limited only by the scope of the claims.

### BRIEF DESCRIPTION OF THE DRAWINGS

A more complete understanding of the present invention may be had by reference to the following Detailed Description and appended claims when taken in conjunction with the accompanying Drawings wherein:

FIG. 1 is a front view of a preferred embodiment of the present invention; and

FIG. 2 is a diagrammatic view of the frequency menu of the present invention; and

FIG. 3 is a diagrammatic view of the frequency menu of the present invention; and

FIG. 4 is a front view of the housing of the present invention containing the graphical interface.

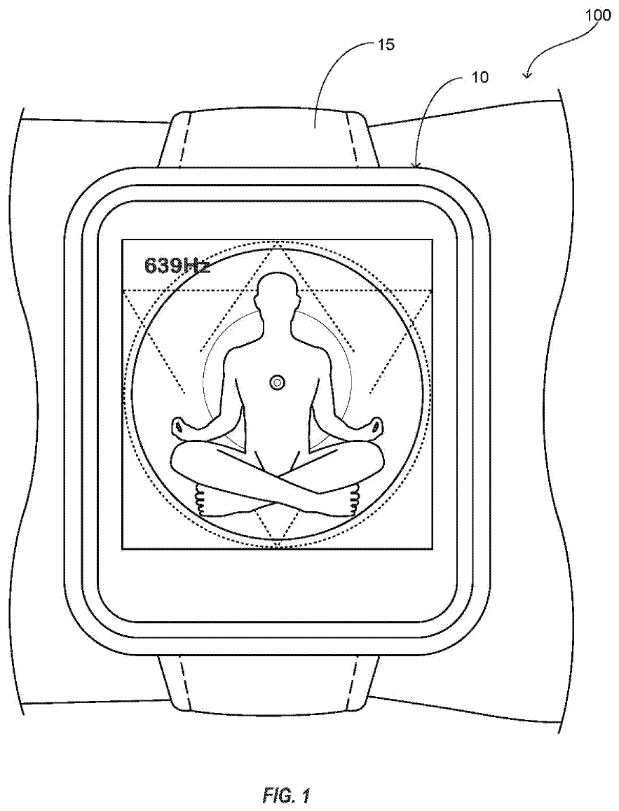

### DETAILED DESCRIPTION

Referring now to the drawings submitted herewith, wherein various elements depicted therein are not necessarily drawn to scale and wherein through the views and figures like elements are referenced with identical reference numerals, there is illustrated a personal frequency apparatus 100 constructed according to the principles of the present invention.

An embodiment of the present invention is discussed herein with reference to the figures submitted herewith. Those skilled in the art will understand that the detailed description herein with respect to these figures is for explanatory purposes and that it is contemplated within the scope of the present invention that alternative embodiments are plausible. By way of example but not by way of limitation, those having skill in the art in light of the present teachings of the present invention will recognize a plurality of alternate and suitable approaches dependent upon the needs of the particular application to implement the functionality of any given detail described herein, beyond that of the particular implementation choices in the embodiment described herein. Various modifications and embodiments are within the scope of the present invention.

It is to be further understood that the present invention is not limited to the particular methodology, materials, uses and applications described herein, as these may vary. Furthermore, it is also to be understood that the terminology used herein is used for the purpose of describing particular embodiments only, and is not intended to limit the scope of the present invention. It must be noted that as used herein and in the claims, the singular forms “a”, “an” and “the” include the plural reference unless the context clearly dictates otherwise. Thus, for example, a reference to “an element” is a reference to one or more elements and includes equivalents thereof known to those skilled in the art. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

References to “one embodiment”, “an embodiment”, “exemplary embodiments”, and the like may indicate that the embodiment(s) of the invention so described may include a particular feature, structure or characteristic, but not every embodiment necessarily includes the particular feature, structure or characteristic.

Referring in particular to the Figures submitted herewith, the personal frequency apparatus 100 includes a housing 10 wherein the housing 10 is manufactured from a durable weatherproof material. The housing 10 is generally square in shape and is configured to be worn on a wrist of a user. The housing 10 is secured to a wrist of a user employing bands 15 wherein it should be understood within the scope of the present invention that the bands 15 could be manufactured from various different materials and provided in numerous alternate colors and styles. The housing 10 has disposed therein the necessary electronics to receive, store, manipulate and transmit data. Furthermore, the housing 10 has disposed therein a power supply that is suitable to provide operation of the software of the present invention. The software of the present invention provides operation of the method of the personal frequency apparatus 100 wherein the user can select a desired chakra frequency as further described herein.

The housing 10 includes a graphical display unit 20 wherein the graphical display unit is configured to provide informational display of the method of the present invention as well as alternate data such as but not limited to the time of day and calendar date. The graphical display unit 20 is operable to provide a technique to operably engage the method of the present invention. It should be understood within the scope of the present invention that the graphical display unit 20 could be provided in alternate sizes and shapes that are similar to that of the housing 10.

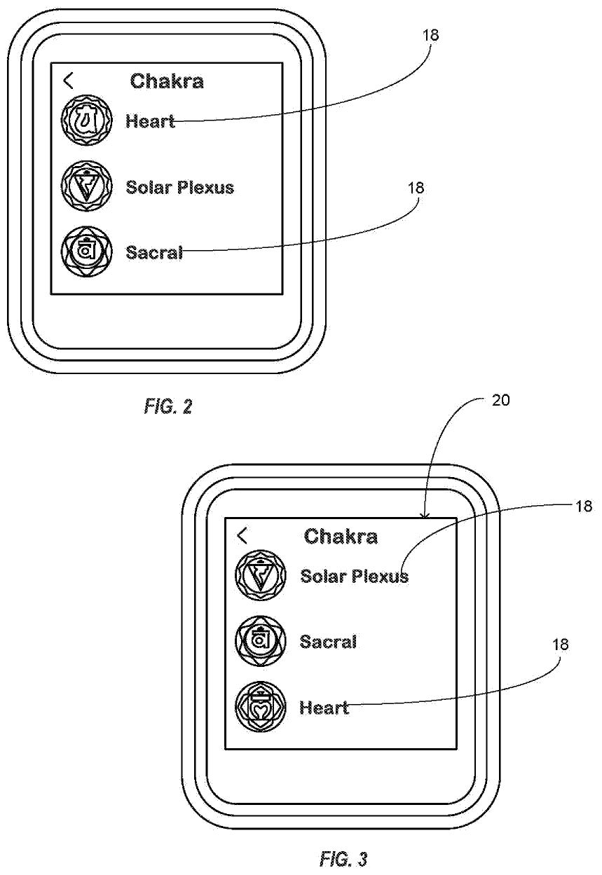

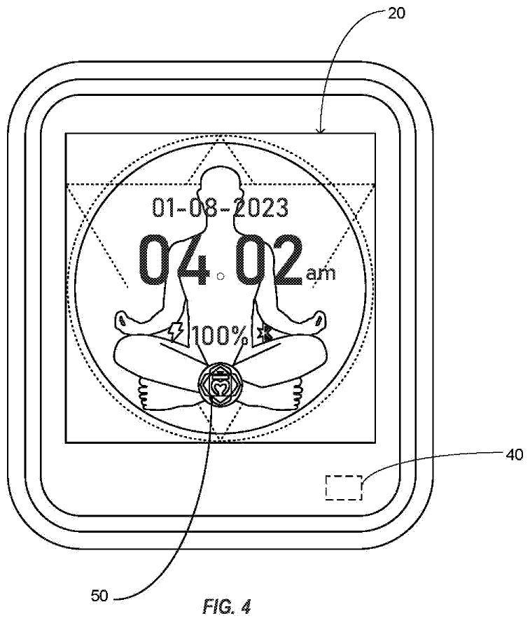

As is illustrated herein in FIG. 2 and FIG. 3, the personal frequency apparatus 100 includes a selection of chakra frequencies 18 via a menu presented on the graphical display unit 20. In use the user will peruse the menu selections of available frequencies and select on frequency from the available choices. The chakra frequencies 18 presented on the graphical display unit 20 represent the primary chakras wherein each are associate with a specific frequency. Selection of one of the chakra frequencies 18 will result in the broadcast of the selected chakra frequency for at least forty-five seconds. The personal frequency apparatus 100 includes a frequency transmission module 40 wherein subsequent selection of a chakra frequency 18 the frequency transmission module 40 will initiate transmission of the frequency for the selected chakra frequency. By way of example but not limitation, if the user selects the heart chakra frequency, the frequency transmission module 40 will initiate and transmit a frequency of 639 Hz. It should be understood within the scope of the present invention that the present invention could provide alternate techniques of frequency generation. By way of example but not limitation, the frequency transmission module 40 could generate the selected frequencies utilizing physical elements within the frequency transmission module 40 or digital synthesis.

The graphical display unit 20 further includes a frequency transmission status indicator 50. The frequency transmission status indicator 50 provides a visual signal that a selected frequency is transmitting. It is contemplated within the scope of the present invention that the personal frequency apparatus 100 could transmit a frequency on demand or provide transmission of frequencies at a specific time interval such as but not limited to hourly. Transmission of the chakra frequency 18 could alternate between the available chakra frequencies or consist of transmission of the same frequency. The graphical display unit 20 includes a frequency transmission indicator 50 that is activated during transmission of a chakra frequency 18. It is contemplated within the scope of the present invention that the chakra frequency 18 transmission occurs for at least forty-five seconds as this is generally equivalent for blood to fully circulate through the human body for a complete cycle.

While the personal frequency apparatus 100 is illustrated and discussed herein as being an apparatus to be worn on the wrist of the user, it is further contemplated within the scope of the present invention that the personal frequency apparatus 100 could be integrated into items such as but not limited to jewelry.

In the preceding detailed description, reference has been made to the accompanying drawings that form a part hereof, and in which are shown by way of illustration specific embodiments in which the invention may be practiced. These embodiments, and certain variants thereof, have been described in sufficient detail to enable those skilled in the art to practice the invention. It is to be understood that other suitable embodiments may be utilized and that logical changes may be made without departing from the spirit or scope of the invention. The description may omit certain information known to those skilled in the art. The preceding detailed description is, therefore, not intended to be limited to the specific forms set forth herein, but on the contrary, it is intended to cover such alternatives, modifications, and equivalents, as can be reasonably included within the spirit and scope of the appended claims.

What is claimed is:

1. A personal frequency transmission apparatus that is configured to provide transmission of a plurality of chakra frequencies wherein the personal frequency transmission apparatus comprises:
a housing, said housing having an interior volume having electronics disposed therein operable to receive, store, transmit and manipulate data, said housing having a graphical display unit, said graphical display unit configured to provide an interface so as to operate software of the present invention, said housing configured to be worn on a body of a user;
a frequency transmission module, said frequency transmission module being disposed within said housing, said frequency transmission module having mechanical and digital elements operable to transmit chakra frequencies.

2. The personal frequency transmission apparatus that is configured to provide transmission of a plurality of chakra frequencies as recited in claim 1, wherein the software of the present invention provides a selection of chakra frequencies to transmit.

3. The personal frequency transmission apparatus that is configured to provide transmission of a plurality of chakra frequencies as recited in claim 2, wherein ensuing selection of a chakra frequency, the frequency transmission module transmits the chakra frequency for at least forty-five seconds.

4. The personal frequency transmission apparatus that is configured to provide transmission of a plurality of chakra frequencies as recited in claim 3, wherein the graphical display unit further includes a portion that is a frequency transmission status indicator, wherein the frequency transmission status indicator is configured to be activated during transmission of the chakra frequency.

5. The personal frequency transmission apparatus that is configured to provide transmission of a plurality of chakra frequencies as recited in claim 4, wherein the housing of the present invention is configured to be secured to a wrist of a user.
